SEQUENCE LISTING (15 sequences)

SEQ ID NO: 1 - DNA, 2722 bp, Artificial Sequence
  IL-60
gttgaaatgaatcggtgtctctcaaagctctatggcaatcggtgtatcggtgtcttacttatacctggacacctaatggctatttggtaatttcataaatgttcattgcaattcaaaattcaaaattcaaaaatcaaatctttaaagcggccatccgtataatattaccggatggccgcgccttttgtttttatgtggtccccacgagggttacacagacgtcactgtcaaccaatcaaattgcattctcaaacgttagataagtgttcatttgtctttatatacttggtccccaagttttttgtcttgcaatatgtgggacccacttcttaatgaatttcctgaatctgttcacggatttcgttgtatgttagctattaaatatttgcagtccgttgaggaaacttacgagcccaatacattgggccacgatttaattagggatcttatatctgttgtaagggccccgtgactatgtcgaagcgaccaggcgatataatcatttccacgcccgtctcgaaggttcgccgaaggctgaacttcgacggcccatacaggcccatgtaccgaaagcccagaaatacagaatgtatcgaagccctgatgttccccgtggatgtgaaggcccctttaaagtccagtcttatgagcaacgggatgatattaagcatcctggtattgttcggttgtgttagtgatgttactcgtggatctggaattactcacagagtgggtaagaggttctgtgttaaatcgatatattttttaggtaaagtctggatggatgaaaatatcaagaagcagaatcacactaatcaggtcatgttcttcttggtccgtgatagaaggccctatggaaacagcccaatggattttggacaggtttttaatatgttcgataatgagcccagtaccgcaaccgtgaagaatgatttgcgtgataggtttcaagtgatgaggaaatttcatgctacagttattggtgggccctctggaatgaaggaacaggcattagttaagagattttttaaaattaacagtcatgtaacttataatcatcaggaggcagcaaagtacgagaaccatactgaaaacgccttgttattgtatatggcatgtacgcatgcctctaatccagtgtatgcaactatgaaaatacgcatctatttctatgattcaatatcaaattaataaaatttatattttatatcatgagtttctgttacatttattgtgttttcaagtacatcatacaatacatgatcaactgctctgattacattgttaatggaaattacaccaagactatctaaatacttaagaacttcatatctaaatactcttaagaaatgaccagtctgaggctgtaatgtcgtccaaattcggaagttgagaaaacatttgtgaatccccattaccttcctgatgttgtggttgaatcttatctgaatggaaatgatgtcgtggttcattagaaatggcctctggctgtgttctgttatcttgaaatagagggggattgttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccctatatgatgaggtaggtcctggattgcagaggaagatagtgggaattccccctttaatttgaatgggcttcccgtactttgtgttgctttgccagtccctctgggcccccatgaattccttgaagtgctttaaataatgcgggtctacgtcatcaatgacgttgtaccacgcatcattactgtacacctttgggcttaggtctagatgtccacataaataattatgtgggcctagagacctggcccacattgttttgcctgttctgctatcaccctcaatgacaatacttatgggtctccatggccgcgcagcggaatacacgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcttcggcatatgcgtcgttggcagattgctgacctcctctagctgatctgccatcgatttggaaaactccaaaatcaatgaagtctccgtctttctccacgtaggtcttgacatctgttgagctcttagctgcctgaatgttcggatggaaatgtgctgacctgtttggtgataccaggtcgaagaaccgttggttcttacattggtatttgccttcgaattggataagcacatggagatgtggttccccattctcgtggagttctctgcaaactttgatgtattttttatttgttggggtttctaggttttttaattgggaaagtgcttcctctttagagagagaacaattgggatatgttaggaaataatttttggcatatattttaaataaacgaggcat

SEQ ID NO: 2 - DNA, 5509 bp, Artificial Sequence
  IL-60-BS
gttgaaatgaatcggtgtctctcaaagctctatggcaatcggtgtatcggtgtcttacttatacctggacacctaatggctatttggtaatttcataaatgttcattgcaattcaaaattcaaaattcaaaaatcaaatctttaaagcggccatccgtataatattaccggatggccgcgccttttgtttttatgtggtccccacgagggttacacagacgtcactgtcaaccaatcaaattgcattctcaaacgttagataagtgttcatttgtctttatatacttggtccccaagttttttgtcttgcaatatgtgggacccacttcttaatgaatttcctgaatctgttcacggatttcgttgtatgttagctattaaatatttgcagtccgttgaggaaacttacgagcccaatacattgggccacgatttaattagggatcttatatctgttgtaagggccccgtgactatgtcgaagcgaccaggcgatataatcatttccacgcccgtctcgaaggttcgccgaaggctgaacttcgacggcccatacaggcccatgtaccgaaagcccagaaatacagaatgtatcgaagccctgatgttccccgtggatgtgaaggcccctttaaagtccagtcttatgagcaacgggatgatattaagcatcctggtattgttcggttgtgttagtgatgttactcgtggatctggaattactcacagagtgggtaagaggttctgtgttaaatcgatatattttttaggtaaagtctggatggatgaaaatatcaagaagcagaatcacactaatcaggtcatgttcttcttggtccgtgatagaaggccctatggaaacagcccaatggattttggacaggtttttaatatgttcgataatgagcccagtaccgcaaccgtgaagaatgatttgcgtgataggtttcaagtgatgaggaaatttcatgctacagttattggtgggccctctggaatgaaggaacaggcattagttaagagattttttaaaattaacagtcatgtaacttataatcatcaggaggcagcaaagtacgagaaccatactgaaaacgccttgttattgtatatggcatgtacgcatgcctctaatccagtgtatgcaactatgaaaatacgcatctatttctatgattcaatatcaaattaataaaatttatattttatatcatgagtttctgttacatttattgtgttttcaagtacatcatacaatacatgatcaactgctctgattacattgttaatggaaattacaccaagactatctaaatacttaagaacttcatatctaaatactcttaagaaatgaccagtctgaggctgtaatgtcgtccaaattcggaagttgagaaaacatttgtgaatccccattaccttcctgatgttgtggttgaatcttatctgaatggaaatgatgtcgtggttcattagaaatggcctctggctgtgttctgttatcttgaaatagagggggattgttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccctatatgatgaggtaggtcctggattgcagaggaagatagtgggaattccccctttaatttgaatgggcttcccgtactttgtgttgctttgccagtccctctgggcccccatgaattccttgaagtgctttaaataatgcgggtctacgtcatcaatgacgttgtaccacgcatcattactgtacacctttgggcttaggtctagatgtccacataaataattatgtgggcctagagacctggcccacattgttttgcctgttctgctatcaccctcaatgacaatacttatgggtctccatggccgcgcagcggaatacacgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcttcggcatatgcgtcgttggcagattgctgacctcctctagctgatctgccatcgatttggaaaactccaaaatcaatgaagtctccgtctttctccacgtaggtcttgacatctgttgagctcttagctgcctgaatgttcggatggaaatgtgctgacctgtttggggataccaagtcgaagaaccgttggttcttacattggtatttgccttcgaattggataagcacatggagatgtggttccccattctcgtggagttctttgcaaactttgatgtattttttatttgttggggtttctagtttttttaattgggaaagtgcttcctctttagagagagaacaattgggatatgttaggaaataatttttggcatatattttaaataaacgaggcatgttgaaatgaatcggtgtccctcaaagctctatggcaatcggtgtatcggtgtcttacttatacttggacacctaatggctatttggtaatttcataaatgttcatttcaattcaaaattcaaaattcaaaaatcaaatcattaaagcggccatccgtataatattaccggatggccgcgccttttccttttatgtggtccccacgagggttacacagatgttattgtcaaccaatcaaattgcattctcaaacgttagataagtgttcatttgtctttatatacttggtccccaagttttttgtcttgcaatatgtgggacccacttcttaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttaggtaaagtctggatggatgaaaatatcaagaagcagaatcacactaatcaggtcatgttcttcttggtccgtgatagaaggccctatggaaacagcccaatggattttggacaggtttttaatatgttcgataatgagcccagtaccgcaaccgtgaagaatgatttgcgtgataggtttcaagtgatgaggaaatttcatgctacagttattggtgggccctctggaatgaaggaacaggcattagttaagagattttttaaaattaacagtcatgtaactttatttatattcattcaggaggcagcaaagtacgagaaccatactgaaaacgccttgttattgtatatggcatgtacgcatgcctctaatccagtgtatgcaactatgaaaatacgcatctatttctatgattcaatatcaaattaataaaatttatattttatatcatgagtttctgttacatttattgtgttttcaagtacatcatacaatacatgatcaactgctctgattacattgttaatggaaattacaccaagactatctaaatacttaagaacttcatatctaaatactcttaagaaatgaccagtctgaggctgtaatgtcgtccaaattcggaagtcgagaaaacatttgtgaatccccattaccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccctatatgaggaggtaggtcctggattgcagaggaagatagtgggaattccccctttaatttgaatgggcttcccgtactttgtgttgctttgccagtccctctgggcccccatgaattccttgaagtgctttaaataatgcgggtctacgtcatcaatgacgttgtaccacgcatcattactgtacacctttgggcttaggtctagatgtccacataaataattatgtgggcctagagacctggcccacattgttttgcctgttctgctatcaccctcaatgacaatacttatgggtctccatggccgcgcagcggaatacacgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcttcggcatatgcgtcgttggcagattgctgacctcctctagctgatctgccatcgatttgggaaactccaaaatcaatgaagtttccgtctttctccacgtaggtcttgacatctgttgagctcttagctgcctgaatgttcggatggaaatgtgctgacctgtttggtgataccaggtcgaagaaccgttggttcttacattggtatttgccttcgaattggataagcacatggagatgtggttccccattctcgtggagttctctgcaaactttgatgtattttttatttgttggggtttctaggttttttaattgggaaagtgcttcctctttagagagagaacaattgggatatgttaggaaataatttttggcatatattttaaataaacgaggcat

SEQ ID NO: 3 - DNA, 314 bp, Artificial Sequence
  IR coding polynucleotide
gttgaaatgaatcggtgtctctcaaagctctatggcaatcggtgtatcggtgtcttacttatacctggacacctaatggctatttggtaatttcataaatgttcattgcaattcaaaattcaaaattcaaaaatcaaatctttaaagcggccatccgtataatattaccggatggccgcgccttttgtttttatgtggtccccacgagggttacacagacgtcactgtcaaccaatcaaattgcattctcaaacgttagataagtgttcatttgtctttatatacttggtccccaagttttttgtcttgcaata

SEQ ID NO: 4 - DNA, 166 bp, Artificial Sequence
  5′ sequence of V2
atgtgggacccacttcttaatgaatttcctgaatctgttcacggatttcgttgtatgttagctattaaatatttgcagtccgttgaggaaacttacgagcccaatacattgggccacgatttaattagggatcttatatctgttgtaagggcccgtgactatgtcg

SEQ ID NO: 5 - DNA, 5742 bp, Artificial Sequence
  Pseudomonas fluorescens tryptophan halogenase PrnA (prnA), PrnB (prnB), halogenase PrnC (prnC) and aminopyrrolnitrin oxidase PrnD (prnD) genes
atgaacaagccaatcaagaatatcgtcatcgtgggcggcggcaccgcgggctggatggccgcttcgtacctcgtccgggcgctccaacagcaggtaaacatcacgctcatcgagtctgcggcgatcccccggatcggcgtgggcgaggcgaccatcccgagtttgcagaaggtgttcttcgacttcctcgggataccggagcgggagtggatgccccaagtgaacggcgccttcaaggccgcgatcaagttcgtgaactggagaaaatctcccgacccatcgcgcgaagattacttctaccatttgttcggcagcgtgccgaactgcgacggcgtgccgcttacccactactggctgcgcaagcgcgaacagggcttccagcagccgatggcgtacgcgtgctatccgcagcccggggccctcgacggcaagctggcaccctgcctggccgacggcacccgccagatgtcccacgcgtggcacttcgacgcgcacctggtggccgacttcttgaagcgctgggccgtcgagcgcggggtgaatcgcgtggtcgacgaggtcgtggaggttcaactgaacgaccgcggctacatctccaccctgttaaccaaggaagggcggacgctggaggcggacctgttcatcgactgctccggcatgcgagggctcctgatcaatcaggccctgaaggaacccttcatcgacatgtccgactacctgctgtgcgacagcgcggtcgccagcgccgtgcccaacgacgacgcgcgcgagggggtcgagccttacacctccgcgattgccatgaactcgggatggacctggaagattccgatgctgggccggttcggcagcggctacgtcttctcgagcaagttcacctcgcgcgaccaggccaccgccgacttcctcaaactctggggcctctcggacaatcagcagctcaaccagatcaagttccgggtcgggcgcaacaagcgggcgtgggtcaacaactgcgtctcgatcgggctgtcgtcgtgctttctggagcccctggaatcgacgggaatctacttcatctacgcggcgctttaccaactcgtgaagcacttccccgacacctcgttcgacccgcggttgcgcgacgcattcaacgccgagatcgtctacatgttcgacgactgccgagacttcgtccaggcgcactatttcactacgtcgcgcgaagacacgccgttctggctcgcgaaccggcacgaactgcggctctcggacgccatccaggagaaggttgagcgctacaaggccgggctgccactgaccaccacctcgttcgacgattccacgtactacgagaccttcgactacgaattcaagaacttctggttgaacggcaactactactgcatctttgccggcctgggcatgctgcccgaccggtcgctgccgctcctgcagcaccgaccggagtcgatccagaaggccgaagcgatgttcgccagcatccggcgcgaagccgagcgcctgcgcacgagcctgccgacgaactacgactacctgcggtcactgcgtgacggcgcgcagctgtcgcgcaaccagcacgggccgacgctcgcggctcaggaacgccagtagtggaacgcaccttgaaccgggtatccgcattcgcggccacacacgctgccgtggcggcctgcgatccgctacaggcacgcgcgctggttctgcagctgccggccctgaaccgtgacaaggacgtgcccggcatcgtcggcctgctgcgcgatttcctcccggtgagcggcgtgccctccagctggggcttcgtcgaagccgccgccgcgatgcgggacatcggtttcttcctggggtcgctcaagcggcacggacatgagcccgtggacctggtgcccgggcttgaacgggtgctgctcgacctggcacgggtgaccgacttgccgccgcgcgagacactcctgcatgtgacggtctggaacccggcggcggccgatgcgcagcggagctacaccgggctgcccgacgaagcgcacctgctcgagagcgtgcgcatctcgatggcggccctcgaggcggccatcgcgttgaccgtcgagctgtccgatgtatccctgcgctcgcccgcgttcgcgcaagggtgcgatgagctggaagcctacctgcagaaaatggtcgaatcgatcgtctacgcgtaccgcttcatctcgccccaggtcttctacgatgagctgcgccccttctacgaaccgattcgagtcgggggccagagctacctcggccccggcgccgtagaaatgcccctcttcgtgctggagcacgtcctgtggggctcgcaatcggacgacccagcttatcgagaattcaaagagacatacctgccctacgtgcttcccgcgtacagggcggtctacgctcggttcgccacaaagccggcgctcatcgaccgtgcgctcgacgaggcgcgagcggtgggtacgcagggcgagcacgtccgggctgggctgacggccctcgagcgggtcttcaaggtcctgctgcgcttccgggcgcctcacctcaaattggcggagcgggcatacgaagccgggcgcagcggccccacaaccggcagcgggggctacgcgcccagcatgctcggcgatctactcacgctcacctgtgccgcgcggtcccgcatccgtgccgcgctcgatgaatcctgatgcgcgcgacccagtgttatctcacaaggagagtttgcccccatgactcagaagagccccgcgaacggacacgatagcaaccacttcgacgtaatcatcctcggttcgggcatgtccggtacccagatgggggccatcctggccaaacaacagtttcgcgtgctgatcatcgagcagtcgtcgcacccgcggttcacgatcggcgaatcgtcgatccccgaaacgtctctcatgaaccgcatcatcgctgatcgctacgacattccggagctcggccacatcacctcgttctactcgacgcagcgttacgtttcgtcgagcacgggcatcaagcgcaacttcggcttcgtgttccacaaacctggccaggagcacgacccgaaggagttcacccagtgcgtcattcccgagctgccgtgggggccggagagccattattaccggcaggacgtcgacgcctatctgttgcaagcggccatcaaatatggctgcacggtccgccagaagacgagcgtgaccgaatatcacgcggacaaggacggcgtcgcggtgaccaccgccgagggcgagcggttcaccggccggtacatgatcgactgcggaggacccggcgcgccgctggcgaccaagttcgggctccgcgaagagccgtgtcgcttcaagacgcactcgcgcagcctctacacgcacatgctcggggtcaagccgttcgacgacatcttcaaggtcaaggggcagcgctggcgctggcacgaaggaaccctgcaccacatgttcaccggcggctggctctgggtgattccgttcaacaaccacccgcgctcgaccaataacctggtgagcgtcggcctgcagctcgacccgcgtgtctacccgaaaaccgacattcccgcgcagcaggaattcgacgagttcctcgcgcggttcccgagcatcggcgctcagttccgggacgccgtgccagtgcgcgactgggtcaagaccgaccgcctgcagttctcgtcgaacgcctgcgtcggcgaccgctactgcctgatgctgcacgcgaacgggttcatcgacccgctcttctcccgggggctcgagaacaccgcggtgaccatccacgcgctcgcggcgcgcctcatcaaggcgctacgcgacgacgacttctcccccgagcgcttcgagtacatcgagcgcctgcagcaaaagcttttggaccacaacgacgacttcgtcagctgctgctacacggcgttctcggacttccgcctatgggacgcgttccaccggctgtgggcggtcggcactatcctcgggcagttccggctggtgcaagcccacgcgaggtttcgcgcgtcgcgcgacgagggcgacctcgatcacctcgacaacgacccgccgtacctcgggtacctgtgcgcggacatggagcagtactaccagttgttcaacgacgccaaagccgaggtcgaggctgtgagcgccgggcacaagtcggccgaggaggccgcgttgcggattcacgccctcatcgacgaacgagacttcgccaagccgatgttcggcttcgggtactgcatcaccggggacaagccgcagctcaacaactcgaagtacagcctgataccggcgatgaagctgatgtactggacgcaaacccgcgcgccggcagaggtgaagaagtacttcgactacaacccgatgttcgcgctgctcaaggcgtacatcaccacccgcatcggcttggctctgaagaagtagtcggccaaggacggcacacacgcgatgaacaacattcaattggatcaagcgaacgtcaagaagcatcccccgggggcgtacgacgcgaccacacgcgtggccgcgagctggtacgtcgcgatgcgctcgaacggcctcagggacaagccgaaggagttgacgctctttggccgtccgtacgtggcgtggcgcgcagcgacggggcaggccgtggtgatggaccgccactgctcgcacctgggcgcgaacctggctgacgggcggatcaaggacgggtgcatccagtgcccgtttcaccactggcgctacgacgagcaaggcaagtgcgttcacatccccggccacagcgaggtggtgcgccagctggagccggtgccacgcgcggcgcgccagccgacgttggtcaccaccgagcgatacggctacgtgtgggtctggtacggctccccgcagccgctgcacccgctgcccgaaatcaccgcagccgacgtcgacaacggcgacttcatgcacctgcacttcgcgttcgagacgacgacggcggtcttgcggatcgtcgagaacttctacgacgcacagcacgcaacccccgtgcacgcgctcccgatctcggccttcgaactcaagctcttcgacgactggagccggtggccggaggttgagtcgctggcccgggcgggcgcgtggttcggtgccgggatcgacttccacgtgaaccgctacttcggccccctcggcatgctgtcgcgcgcgctcggcctgaacatgtcgcagatgaacctgcacttcgatggctaccccggcgggtgcgtcatgaccgttgccctggacgcagacgtcaaatacaaactgctccagtgtgtgacaccggtgagcgacggcaagaacatcatgcacatgctcatctcgatcaagaaggtgggcggcgtcctgcgccgtgcgaccgacttcgtgctgttcgggctgcagaccagacaggcagcggggtacgacgtcaaaatctggaacgggatgaagcccgacggcggcggcgcttacagcaagtacgacaagctcgtgctcaagtaccgtgcgttctaccgcggctgggtcgaccgtgtcgcgagtgagcagtaatgcgtgaggccgagccggtagcggtcgcgtcgcgctgcccggcgcttgcgaacctttcgagctgcgtcacggagatcacggcgtacggcgcggcgggcccgcttgggctcgcggccacccgcttggtgtcggtgtcgctctttgcgaggtattgatgaccatctggctgttgcaactcgtg

SEQ ID NO: 6 - DNA, 30 bp, Artificial Sequence
  Primer sequence
gccagatagtcatgaatacctcgcaaagag

SEQ ID NO: 7 - DNA, 36 bp, Artificial Sequence
  Primer sequence
atggatccatgaacaagccaatcaagaatatcgtca

SEQ ID NO: 8 - DNA, 20 bp, Artificial Sequence
  Primer sequence
ggccccagcatcggaatctt

SEQ ID NO: 9 - DNA, 20 bp, Artificial Sequence
  Primer sequence
gcgaacgaacacgatagcaa

SEQ ID NO: 10 - DNA, 19 bp, Artificial Sequence
  Primer sequence
cgtcaatgagggcgtgaat

SEQ ID NO: 11 - DNA, 17 bp, Artificial Sequence
  Primer sequence
atgaacaacttcaattg

SEQ ID NO: 12 - DNA, 30 bp, Artificial Sequence
  Primer sequence
attctagacacgagttgcaacagccagatg

SEQ ID NO: 13 - DNA, 24 bp, Artificial Sequence
  Primer sequence
atgagtaaaggagaagaacttttc

SEQ ID NO: 14 - DNA, 24 bp, Artificial Sequence
  Primer sequence
ttaaagctcatcatgtttgtatag

SEQ ID NO: 15 - DNA, 30 bp, Artificial Sequence; identical to SEQ ID NO: 6
  Primer sequence

(19) United States
(12) Patent Application Publication
(10) Pub. No.: US 2014/0325707 A1
(43) Pub. Date: Oct. 30, 2014

(54) GROWTH ENHANCEMENT OF PLANTS

(71) Applicant: Yissum Research Development Company of the Hebrew University of Jerusalem Ltd., Jerusalem (IL)

(72) Inventors: Ilan SELA, Ramot-HaShavim (IL); Haim David Rabinowitch, Kiryat-Ono (IL); Leonid Chernin, Rehovot (IL); Rita Mozes-Koch, Yavne (IL)

(73) Assignee: Yissum Research Development Company of the Hebrew University of Jerusalem Ltd., Jerusalem (IL)

(21) Appl. No.: 14/225,823

(22) Filed: Mar. 26, 2014

Related U.S. Application Data
(63) Continuation-in-part of application No. PCT/IL2012/050386, filed on Sep. 24, 2012.
(60) Provisional application No. 61/539,046, filed on Sep. 26, 2011.

(51) Int. Cl. C12N 15/82

(52) U.S. Cl. CPC C12N 15/8261
USPC 800/290; 800/298

(57) ABSTRACT

The present invention provides means and methods for producing plants with enhanced growth and yield. Particularly, the present invention provides plants to which an expression construct comprising the entire prn operon was introduced, wherein said plants produce pyrrolnitrin and show enhanced growth and yield production compared to corresponding control plants not harboring the prn operon.

### RELATED APPLICATION

This application is a Continuation in part (CIP) of PCT Patent Application PCT/IL2012/050386, having International filing date of Sep. 24, 2012, which claims the benefit of priority under 35 USC §119(e) of U.S. Provisional Patent Application No. 61/539,046, filed on May 25, 2011, the contents of which are incorporated herein by reference in their entirety.

### FIELD OF THE INVENTION

The present invention provides means and methods for producing plants with enhanced growth and yield, particularly to means and method for producing plants expressing the complete prn operon that show enhanced growth and yield production compared to corresponding plants lacking the prn operon.

### BACKGROUND OF THE INVENTION

Prokaryotic genes are usually clustered in operons under the control of a common promoter, and proteins are translated from a polycistronic mRNA. Eukaryotic proteins are translated from their generally monocistronic mRNAs in a 5′-dependent manner. Nevertheless, evidence show that in some cases, eukaryotic genes associated with functionally related activities are also clustered together and co-expressed.

Gene clustering may also result in co-regulation of gene expression, and in some cases, genes participating in a certain metabolic pathway are clustered and co-regulated. However, gene clusters do not always share a common cis-element, such as a promoter, and thus are not always co-regulated by translation from the same polycistronic mRNA. Several cis-controlled clusters have arisen from gene duplication and divergence, e.g. the human β-globin gene cluster. However, such clusters represent homologs of the same ancestral gene and not diverse genes requiring coordinated regulation for a particular metabolic pathway. On the other hand, heterologous gene clusters participating in the same metabolic pathway have been found in plants. Clustering indicates the possibility of co-segregation (preserving the clusters upon meiosis and recombination). However, despite the “operon-like” clustering and co-expression of pertinent genes (Field B and Osbourn A. E., 2008. Science, 320:543-547), expression from a single transcription unit, such as the classical operon, has not yet been demonstrated in eukaryotes. The current dogma is that in most cases, co-regulation of eukaryotic gene clusters is due to chromosomal arrangements and chromatin potentiation, which affects an entire chromosomal domain (e.g. Kingston R E and Narlikar G J., 1999. Genes Dev 13:2339-2352; Gierman H J et al., 2007. Genome Res 17:1286-1295). Cases in which expression of dicistronic mRNAs is regulated by read through of a stop codon have also been reported. Operon-like gene organization has been found in nematodes, yeast (Saccharomyces cerevisiae), and some higher eukaryotes. However, the term “operon-like” may not be accurate in all cases; “clustered organization” better describes the various situations. Nematodes and Drosophila carry gene clusters that are transcribed to polycistronic pre-mRNAs and on a structural basis can be defined as operon like (summarized in Blumenthal T., 2004. Brief Fun Genomics and Proteomics 3:199-211). However, unlike in prokaryotes, the polycistronic pre-mRNAs are processed to monocistronic (sometimes dicistronic) mature mRNAs and are co-regulated by virtue of being transcribed from the same promoter. Intergenic regions (IRs) are removed by splicing and open reading frames (ORFs) are condensed to form a number of mRNAs. The resultant monocistronic mRNAs are trans-spliced to SL2 (a species of small nuclear RNA), which carries a cap structure, enabling 5′ initiation of translation from each individual monocistronic structure.

Several rhizospheric bacteria produce antifungal and antibacterial secondary metabolites, and their use as biocontrol agents of soil-borne plant pathogens has been attempted. Arima et al. (Arima K et al., 1964. Agric. Biol. Chem. 28:575-576) were the first to our knowledge to report the antibiotic compound 3-chloro-4-(2′-nitro-3′-chlorophenyl)-pyrrole (pyrroInitrin [PRN]) produced by a number of Pseudomonas pyrrocinia strains. PRN has been found active against a wide range of pathogens and is produced by other bacterial species as well, including Pseudomonas fluorescens, Burkholderia cepacia, and Serratia plymuthica. A four-gene operon coding for an enzymatic pathway converting Trp to PRN has been identified (Kirner et al., 1998) and the function of every encoded protein determined (Kirner S et al., 1998. J Bacteriol 180:1939-1943). No significant homology has been found between the first three enzymes (PrnA, PrnB, and PrnC) and any plant protein. However, pheophorbide α-deoxygenase (synonymous with ACD1 and LLS1) from several plants shows 42% to 48% similarity to PrnD.

The biosynthesis of a secondary metabolite involves numerous enzymes, the genes of which constitute a metabolic pathway. Attempts to introduce multiple genes into plants by various techniques (e.g. gene stacking) have been somewhat successful (Halpin, C., 2005. Plant Biotechnol J 3:141-155). However, lack of co-regulation remains the main obstacle in this latter technique. The best method to date for metabolic engineering is plastid transformation. Because plastids are of prokaryotic origin, they can express several genes from a single polycistronic mRNA. The ability to express a string of co-regulated genes could potentially result in activation of an entire metabolic pathway and the production of its end product, a nonproteinaceous secondary metabolite (Wang H H et al., 2009. Genome 36:397-398). However, for this to happen, an operon and its regulatory elements have to be artificially constructed, and concerted regulation and optimization of the stoichiometry of the various components have yet to be achieved.

IL-60 is a platform of constructs derived from the Geminivirus Tomato yellow leaf curl virus (TYLCV). The IL-60 system has provided universal expression or silencing in all plants tested to date (Peretz Y et al., 2007. Plant Physiol 145:1251-1263; International Application Publication No. WO 2007/141790).

The WO 2007/141790 Application describes use of the IL-60 system to mediate the introduction and expression of an entire bacterial operon in tomato (Solanum lycopersicum) plants. The operon was transcribed and translated in the plant in a manner conforming to that of classical bacterial operons. Expression of the entire pathway resulted in the appearance of a unique secondary metabolite (PRN).

International Application Publication No. WO 2010/004561 discloses methods of expressing a molecule of interest in a plant. The method comprises contacting roots of the plant in a solution comprising at least one Geminivirus based expression construct, particularly the IL-60 system so as to allow construct to be absorbed by the roots and further to be spread systemically and in the plant host without showing disease symptoms.

International Application Publication No. WO 2011/001434 provides means and methods for simple and efficient introduction of foreign genetic material into the plant cell. Particularly, the invention combines seed priming and Geminivirus-based DNA constructs, particularly the IL-60 system, for efficient introduction of heterologous DNA into plants.

Crop production is affected by numerous biotic and abiotic environmental factors. The increase in demand for higher crop productivity due to economic reasons, declining availability in means for production (soil/water), the increase in demand (for example in China and India) and in the world population thus requiring the development of sophisticated agricultural methods as well as of highly productive crop plants.

Thus, there is a recognized need and it would be highly advantages to have means and methods for improving the growth and yield of crop plants.

### SUMMARY OF THE INVENTION

The present invention answers the above-described need by providing plants expressing the complete prn operon which shows enhanced growth and yield production compared to reference control plants.

The present invention is based in part on the unexpected discovery that plant into which the entire prn operon was introduced expressing its end product pyrroInitrin (PRN) are not only resistant to diseases caused by pathogenic bacteria, but also show improved growth rate under optimal and suboptimal conditions, leading to improved growth and crop yield production.

Thus, according to one aspect, the present invention provides a method for enhancing the growth of a crop plant, comprising (a) introducing into at least one cell of a plant at least one virus-based expression construct comprising heterologous DNA comprising the entire prn operon thereby enhancing the growth of the plant compared to the growth of a corresponding control plant.

According to certain embodiments, the virus-based expression construct comprises viral genes or parts thereof enabling replication and symptomless spreading of the construct into adjacent plant cells throughout the entire plant.

According to typical embodiments, the expression construct is a Geminivirus based construct. According to these embodiments, the construct comprises the entire prn operon downstream to non-contiguous nucleic acid sequences of Geminivirus intergenic region (IR).

According to certain embodiments, the Geminivirus is Tomato Yellow Leaf Curl Virus (TYLCV).

According to certain embodiments, the method of the present invention comprises introducing into the at least one plant cell a single TYLCV based expression construct comprising the entire prn operon, wherein the construct enables replication and symptomless spreading of said construct into adjacent plant cells. According to certain embodiments, the TYLCV based expression construct is selected from the group consisting of IL-60 having the nucleic acids sequence set forth in SEQ OD NO:1 and IL-60-BS having the nucleic acids sequence set forth in SEQ ID NO:2.

According to other embodiments, the method of the present invention comprises co-introducing into the plant cell a first TYLCV based expression construct comprising the entire prn operon and viral genes or parts thereof enabling replication confined to the plant cell and a second TYLCV based expression construct comprising viral genes or parts thereof enabling symptomless spreading of at least the first construct from said plant cell into adjacent plant cells and throughout the entire plant.

According to these embodiments, the first DNA construct comprises the entire prn operon downstream of a non-contiguous TYLCV viral intergenic region (IR).

According to certain typical embodiments, the first expression constructs comprises an intergenic region (IR) of TYLCV followed by the 5′ terminal sequence of TYLCV V2 upstream of the entire prn operon, said expression construct is designated IR-PRN. According to certain embodiments, the intergenic region (IR) of TYLCV has the nucleic acid sequence set forth in SEQ ID NO:3 and the 5′ terminal sequence of TYLCV V2 has the nucleic acid sequence set forth in SEQ ID NO:4.

According to further typical embodiments, the second expression construct is selected from the group consisting of IL-60 having the nucleic acids sequence set forth on SEQ OD NO:1 and IL-60-BS having the nucleic acids sequence set forth in SEQ ID NO:2.

According to certain embodiments, the prn operon encodes pyrroInitrin (3-chloro-4-(2′-nitro-3′-chlorophenyl, PRN). According to some embodiments, the prn operon is the entire prn operon of Pseudomonas fluorescens strain Pf-5 (GenBank accession No. CP000076.1; bases 4,157,074-4,162,815). According to certain embodiments, the prn operon comprises the nucleic acid sequence set forth in SEQ ID NO:5.

The construct of the present invention is designed as an expression construct such that the heterologous PRN encoding DNA is expressed in the plant cell. The construct may thus further comprise at least one regulatory element selected from the group consisting of an enhancer, a promoter, and a transcription termination sequence.

Any crop plant can be used according to the teachings of the present invention. According to certain embodiments, the host plant is selected from the group consisting of Solanaceae, Cucurbitaceae, Umbelliferae, Liliacae, Gramineae (Poaceae), Rosaceae, Musaceae, Vitacea, and Cruciferae.

According to additional embodiments, the virus-based expression construct further comprises a marker for identifying the seeds and/or plants comprising the heterologous PRN encoding DNA.

The Geminivirus-based expression construct of the present invention is so designed such that the prn operon polynucleotide is not incorporated into the cell genome. The introduced construct is capable of replicating and spreading within the cells of the plant.

Introducing the construct of the invention may be performed by various means, as is known to one skilled in the art. Common methods are exemplified by, but are not restricted to, Agrobacterium-mediated DNA introduction, microprojectile bombardment, pollen mediated transfer, liposome mediated introduction, direct gene transfer (e.g. by microinjection) and electroporation of compact embryogenic calli.

According to certain embodiments, the method further comprises analyzing the amount of crop yield or biomass of the plant.

According to certain embodiments, the analyzing is effected by measuring an expression of a growth-related gene.

According to certain embodiments, the analyzing is effected by measuring a weight or a volume of said crop yield of the plant.

According to certain embodiments, the analyzing is effected by measuring a weight or a number of seeds of the plant.

According to certain embodiments, the growth of the plant is effected in the absence of a bacterial or fungal contaminant sensitive to said PRN.

According to certain currently preferred embodiments the at least one Geminivirus, particularly TYLCV based expression constructs of the invention is introduced into the at least one plant cell by placing roots of said plant in a solution comprising the at least one Geminivirus-based expression construct so as to allow said at least one Geminivirus-based expression construct to be introduced into the root cells. In alternative embodiments, the at least one construct is introduced into a plant seed embryo by contacting (e.g. soaking) a plant seed which has not undergone imbibitions with a priming medium containing said at least one Geminivirus-based expression construct under conditions enabling priming. These methods of introducing the expression constructs of the invention are highly advantageous as they are highly efficient, easy to perform and do not cause damage to the cell. Furthermore, the prn polynucleotide is directly introduced into a rooted plant or a plant seed embryo that can develop to an entire plant. The symptomless spreading capability of the Geminivirus-based expression construct further ensures that a large number of plant cells express the desired PRN.

According to additional aspect, the present invention provides a plant comprising at least one cell comprising at least one Geminivirus based expression construct, the construct comprising an entire prn operon downstream to a non-contiguous nucleic acid sequences of Geminivirus intergenic region (IR), wherein the plant shows enhanced growth compared to a corresponding control plant.

According to certain embodiments, the Geminivirus-based construct is selected from the group consisting of IL-60 having the nucleic acids sequence set forth on SEQ OD NO:1 further comprising the prn operon; IL-60-BS having the nucleic acids sequence set forth in SEQ ID NO:2 further comprising the prn operon; IR-PRN comprising the nucleic acid sequence set forth in SEQ ID NOs; 3 and 4, further comprising the prn operon; and combinations thereof.

According to other embodiments, the prn operon encodes pyrroInitrin (3-chloro-4-(2′-nitro-3′-chlorophenyl, PRN). According to some embodiments, the prn operon is the entire prn operon of Pseudomonas fluorescens strain Pf-5 (GenBank accession No. CP000076.1; bases 4,157,074-4,162,815). According to certain embodiments, the entire prn operon comprises the nucleic acid sequence set forth in SEQ ID NO:5.

As used herein, the terms “a plant having enhanced growth” or “a plant showing enhanced growth” or “a plant having improved growth response” refer to a plant with at least one of increased height, fresh weight, dry weight, any desired commercial phenomenon or combinations thereof compared to a corresponding control plant not harboring the prn operon according to the teachings of the present invention.

According to certain embodiments, the growth of the PRN expressing plant is enhanced by at least 30% or more compared to the growth of the corresponding reference control plant.

According to certain embodiments, the plants of the present invention expressing the prn operon are further characterized by enhanced crop yield compared to a corresponding plant that does not comprise the prn operon. According to further embodiments, the plants expressing the prn operon are further characterized by increased amounts of nutritional components. According to these embodiments, the crop plant is selected from the group consisting of plants producing fruit; flower and ornamental plants; grain producing plants i.e., cereal crops (wheat, oats, barley, rye, rice, maize sorghum, Eragrostis and other species including interspecific plants such as Triticale); other grain producing plants as Amaranthus); legumes (peanuts, peas soybean lentil etc); forage crops used for hay or pasture; root crops (sweet potatoes etc), fiber crops (cotton, flax etc); trees for wood industry; tuber crops (potato), sugar crops (sugar beet, sugar came), oil crops (canola, sunflower, sesame, rapeseed etc), green-fuel producing crops (as castor, jatropha, rapeseed), wherein each possibility represents a separate embodiment of the invention.

According to yet additional aspect, the present invention provides a Geminivirus based expression construct capable of replication confined to a host cell, the construct comprises the Geminivirus intergenic region (IR) and the 5′ sequence of the Geminivirus V2, said construct is devoid of the viral coat proteins.

According to certain embodiments, the Geminivirus is Tomato Yellow Leaf Curl Virus (TYLCV). According to these embodiments, the intergenic region comprises the nucleic acid sequence set forth in SEQ ID NO:3 and the 5′ sequence of V2 comprises the nucleic acid sequence set forth in SEQ ID NO:4.

Other objects, features and advantages of the present invention will become clear from the following description and drawings.

### BRIEF DESCRIPTION OF THE FIGURES

FIGS. 1A-1D schematically illustrates (not to scale) the constructs used in this study, and the prn operon. All constructs were circular. FIG. 1A: IR-PRN. prn operon fused to TYLCV's IR within the plasmid pDrive. FIG. 1B: IL-60-BS. C1→C4 and V1, V2 are ORFs of TYLCV. Note that C1 and C4 are interrupted by the plasmid Bluescript. FIG. 1C: GFP fused to the PRN operon. pD: the plasmid pDrive. BS: The plasmid Bluescript. FIG. 1D: The prn operon illustrating the positions of PCR primers (primer numbers below the arrows refer to Table 1).

FIGS. 2A-2B show representative analysis for the presence of prn genes in treated plants. FIG. 2A: DNA was extracted from systemic leaves and subjected to PCR using different sets of primers (Table 1). Amplification of: a prnA fragment (lane 1), TYLCV-IR (lane 2), a prnB and prnC-spanning fragment (lane 3), and a prnD fragment (lane 4). FIG. 2B: Presence of prnA in various tomato organs. Amplification with prnA primers of DNA extracted from roots (lane 1), leaves (lane 2), fruit (lane 3), leaves of an untreated plant (lane 5), and without template (lane 6). Lanes 4 and 7 are empty. M: Size marker.

FIG. 3 shows RT-PCR analysis of the prn operon transcription. The amplified sequence is a segment of prnD. RT-PCR of the same plant RNA amplifying a fragment of tomato actin is depicted at the bottom. M: Size marker. Lanes 1 to 8, Template RNA extracted from various prn-carrying plants. Lane 9, Control; RNA was extracted from reference plant not harboring the prn operon.

FIG. 4 demonstrates the biological activity of various HPLC fractions of root extract obtained from PRN-expressing plants. Each plate contains a disk of agar with Rizoctonia solani mycelium and addition of (left to right) fractions eluted between 1 and 3 min; fractions eluted between 3 and 5 min; fractions eluted between 5 and 25 min; fractions eluted between 30 and 32 min; and synthetic PRN (0.2 μg/plate).

FIG. 5 shows the expression of GFP in tomato plants harboring the IR-PRN-GFP construct. Plant samples were taken 2 weeks after the construct was introduced and examined under a confocal microscope. Panel A: Picture taken after excitation for GFP fluorescence. Panel B: Picture taken with a filter that masks GFP fluorescence (showing chlorophyll autofluorescence). Panel C: Picture taken without excitation. Panel D: Superposition of panels A and B.

FIGS. 6A-6B demonstrate that PRN-expressing plants are resistant to damping-off disease. FIG. 6A: the construct IR-PRN was introduced into tomato seedlings which were then planted in non-infested soil. The plants were transferred to R. solani-infested soil one week after administration of IR-PRN. The picture was taken 2 weeks after R. solani infestation. A PRN-expressing plant is shown on the left and a reference control plant on the right. FIG. 6B: IR-PRN was administered to tomato seedlings which were immediately planted in R. solani-infested soil. The group of plants on the right is expressing PRN, and the group on the left consists of reference control plants. The picture was taken 5 days after planting.

FIGS. 7A-7B demonstrate that IR-PRN is expressed as a long transcript. FIG. 7A shows Northern-blot analyses of RNA from IR-PRN-harboring plant (lane 1) and reference control plant (lane 2). A segment of prnA served as a probe. FIG. 7B shows Long-distance PCR of cDNA reverse transcribed from ribosome-bound RNA. Primers for RT and PCR were from both ends of the prn operon (Lane 3). M: Size marker.

FIGS. 8A-8B show the effect of PRN on the performance of the plants. The height, fresh weight and dry weight of tomato seedlings to which the entire prn operon has been introduced were compared to those of reference control seedlings (FIG. 8A). The overall growth enhancement is shown in FIG. 8B.

### DETAILED DESCRIPTION OF THE INVENTION

The present invention relates to plants comprising the entire prn operon and expressing its end product PRN that are characterized by enhanced growth, leading to enhanced crop yield.

### Definitions

The term “plant” is used herein in its broadest sense. It includes, but is not limited to, any species of the Gymnospermae and Angiospermae, monocots and dicots, woody, herbaceous, perennial or annual plant. It also refers to a plurality of plant cells that are largely differentiated into a structure that is present at a stage of the plant development capable of producing crop.

As used herein, the term “crop plant” refers to a plant with at least one part having commercial value. The term encompasses plants producing edible fruit (including vegetables), plants producing grains (as a food, feed and for oil production), plant producing flowers and ornamental plants, legumes, root crops, tuber crops, leafy crops, plants grown for timber, fibers, volatiles, resins, pigments and any other metabolites (e.g. compounds with medical value for example, salicilic acid, vincristine, ephedrine; plant producing nutraceutical compound, including vitamins, allicin, and the like.

The term “enhanced growth” as used herein refers to an increase in at least one of plant height, fresh weight, dry weight, any desired commercial phenomenon (e.g. odor) or any combination thereof as compared to these parameters measured for corresponding reference control plant not harboring the prn operon and grown under the same environmental conditions.

The term “increased yield” as used herein refers to an increase in the overall production of the commercially valuable plant part or trait. The term encompasses increase in the plant part mass, number or both.

Methods of analyzing an increased growth of the plant are known in the art and include measuring various attributes of the plant such as measuring height, weight (dry and/or wet) and or measuring for expression of a growth related gene of the plant.

Other parameters which may be analyzed include analyzing the crop yield of the plant. This may be effected by weighing the crop yield or measuring the height or volume of the crop yield. Alternatively, the number of seeds may be counted per plant or per growth area. Other measurements which may be made include seed volume per plant or per growth area, number of seeds per plant or per growth area, seed weight per plant or per growth area.

As used herein, a plant growth related gene is a gene that plays a role in determining growth rate, overall size, tissue size, or tissue number of a plant. Such growth related genes may be identified when modification of their function by mutation, overexpression, or suppression of expression results in altered plant growth rate, overall plant size, tissue size or number, or altered development. Plant and growth related genes can exert their effects through a number of mechanisms some of which include regulation of cell cycle, plant hormone synthesis/breakdown pathways, sensitivity to plant hormones, cell wall biosynthesis, cell identity determination, and the like. Some examples of plant growth related genes are disclosed in U.S. Application No. 20080263727, the contents of which is incorporated herein by reference.

The term “prn operon” as used herein refers to polynucleotide comprising all our gene encoding the enzymatic pathway converting tryptophan (Trp) to pyrroInitrin (3-chloro-4-(2′-nitro-3′-chlorophenyl, PRN). The enzymes are PrnA, PrnB, PrnC and PrnD. According to some embodiments, the prn operon is the entire prn operon of Pseudomonas fluorescens strain Pf-5 (GenBank accession No. CP000076.1; bases 4,157,074-4,162,815).

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

The terms “heterologous DNA” or “exogenous DNA” refer to a polynucleotide that is not present in its natural environment (i.e., has been altered by the hand of man). In the context of the present invention the term heterologous DNA refers to a polynucleotide from one species introduced into another species, particularly to polynucleotide encoding bacterial enzymes introduced into plants.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments.

As used herein, the term an “enhancer” refers to a DNA sequence which can stimulate promoter activity, and may be an innate element of the promoter or a heterologous element inserted to enhance the level or tissue-specificity of a promoter.

The term “expression”, as used herein, refers to the production of a functional end-product e.g., an mRNA, a protein or any other secondary metabolite.

The terms “a plant cell comprising”, or “a plant comprising” or a “plant cell harboring” or “a plant harboring” the virus-based expression construct as used herein include the cell or cells into which the virus-based construct according to the teachings of the present invention was introduced as well as to adjacent cells into which the construct has been spread; cultures derived from these cells regardless to the number of transfers and plants developed therefrom. All progeny of each cell into which the construct was introduced may not be precisely identical in DNA content, due to deliberate or inadvertent mutations.

The terms “control plant” “reference plant” or “reference control plant” are used herein interchangeably referring to a plant not harboring an expression constructs according to the teachings of the present invention, being of the same species of the plant comprising the construct, of the same cultivar and of the same seed lot.

Introduction of a foreign DNA into a cell may result in stable or transient introduction of the DNA. The term “transient introduction” or “transiently introduced” refers to the introduction of one or more exogenous polynucleotides into a cell in the absence of integration of the exogenous polynucleotide into the host cell's genome. Transient introduction may be detected by, for example, enzyme-linked immunosorbent assay (ELISA), which detects the presence of a polypeptide encoded by one or more of the exogenous polynucleotides. Alternatively, transient introduction may be detected by detecting the activity of the protein (e.g. β-glucuronidase) encoded by the exogenous polynucleotide or by other trait conferred by the expression of the transiently introduced DNA.

The virus based constructs of the present invention may comprise viral elements (e.g. genes) from either DNA or RNA viruses. As used herein the phrase “DNA virus” refers to a virus with DNA as its genetic material and replicates using a DNA-dependent DNA polymerase. The DNA virus may belong to either Group I (double-stranded DNA; dsDNA) or Group II (single-stranded DNA; ssDNA) of the Baltimore classification system for viruses. Single-stranded DNA is usually expanded to double-stranded in infected cells.

Examples of single stranded DNA viruses which infect plants are those that belong to the Geminiviridae or Nanoviridae family. According to one embodiment, the geminivirus based expression construct is a begomovirus (e.g. bean golden mosaic virus, beet curly top virus, maize streak virus, tomato pseudo-curly top virus, Bean Yellow Dwarf Virus (BeYDV) and tomato yellow leaf curly virus).

Examples of RNA plant viruses contemplated by the present invention include brome mosaic virus (BMV) barley stripe mosaic virus (BSMV), Tobacco Mosaic Virus (TMV), Potato Virus X (PVX), and Cowpea Mosaic Virus (CPMV).

According to typical embodiments of the present invention, the virus based DNA construct of the invention, particularly the Geminivirus based DNA construct is transiently introduced into the plant. Cells and/or plants into which a DNA construct is transiently introduced are typically referred to as “non-transgenic” or “non-genetically modified (non-GMO)”.

### Preferred Modes for Carrying Out the Invention

The present invention provides method for enhancing the growth of plants and to plants so produced. Particularly, the plants are crop plants, and thus the growth enhancement results in an increase in the crop yield and/or in any desirable commercial trait.

According to one aspect, the present invention provides a method for enhancing the growth of a crop plant, comprising introducing into at least one cell of a plant at least one virus-based expression construct comprising heterologous DNA comprising the entire prn operon thereby enhancing the growth of the plant compared to the growth of a corresponding control plant.

According to certain embodiments, the virus-based expression construct comprises viral genes or parts thereof enabling replication and symptomless spreading of the construct from the plant cell to which it was introduced into adjacent plant cells.

The virus-based DNA construct of the present invention is capable of systemic, symptomless spread throughout the plant to which it was introduced. As used herein, the term “systemic, symptomless spread” refers to the ability of the virus-based vector to spread, for example, from cells of the roots to the foliage, from cells of the seed embryo to the entire plant and to the developing leaf cells, without inducing the characteristic pathogenic symptoms of the virus.

According to certain embodiments, the method of the present invention comprises introducing into the plant cell a single expression construct based on the Geminivirus genetic components. The Geminivirus based expression construct, designated IL-60, is disclosed in International (PCT) Application Publication No. WO2007/141790 and further described by Peretz et al. (2007, ibid). The construct comprises the heterologous polynucleotide sequence, the entire prn operon according to the teachings of the present invention, downstream, in a non-contiguous manner, to the nucleic acid sequences of the Geminivirus intergenic region (IR).

According to other embodiments, the method of the present invention comprises co-introduction into the plant cell an IL-60 based expression construct with additional Geminivirus based expression construct, designated IR-PRN, comprising the viral intergenic region (IR) upstream the heterologous polynucleotide sequence, the entire prn operon according to the teachings of the present invention. Geminivirus-based expression constructs comprising IR upstream the heterologous polynucleotide sequence are described in U.S. Patent Application Publication No. 2011/0162106, incorporated herein by reference.

Co-administration of IL-60 based expression construct and Geminivirus-based construct comprising IR and the molecule of interest is also disclosed in WO2007/141790 and U.S. 2011/0162106.

The IL-60 system provides a universal expression or silencing tool for all plants tested to date. The present invention now shows that expression of a complete bacterial prn operon in tomato by co-administration of IL-60-BS and IR-PRN resulted in enhanced growth of the tomato plants, as reflected at least by enhanced height and weight. The prn operon (around 6 kb, SEQ ID NO:5) of strain Pf-5 of P. fluorescens encodes the broad-range antifungal and antibacterial PRN. The TYLCV-based expression system provides for the delivery and expression of this rather large bacterial operon in plants. Expression of the prn operon in plants leads to the appearance of a major unique metabolic trait manifested in the ability to produce a functionally active secondary metabolite (PRN). It is further shown that the production of PRN increased the resistance of the plants to Fusarium crown root rot disease caused by the fungus Fusarium oxysporum f. sp. radicis-lycopersici (FORL) and to root rot caused by the fungus Rizoctonia solani used as model plant pathogens.

Secondary metabolites are non proteinaceous compounds that contribute to the molecular programs required for normal growth and development in plants including contribution to the defense mechanisms of the plant, elimination of waste products etc. They serve as mediators in many metabolic pathways and contribute to plant interactions with the environment, some of which are modulated by plant hormones. Pigments and fragrance attract pollinators and are therefore essential for plant reproduction. At the same time, plant volatiles serve as repellents to herbivores, and, after infestation by a pest, alarm pheromones are emitted that attract natural enemies of that pest. Pigments also function as strong anti-oxidants. Plant secondary metabolites also participate in the elicitation of induced resistance to pathogens and pests. Plants produce a plethora of secondary metabolites that are major ingredients in a wealth of potentially economically valuable substances such as pharmaceuticals, food additives, fragrances, natural pesticides, and more. However, the production of these metabolites from plants and plant cultures has not yet reached a complete transition from economic potential to commercial success (Zhang W et al., 2004. J Biomed Biotechnology 2004:264-271). Numerous approaches have been attempted to improve the yield of a desired metabolite to a commercially significant level; metabolic engineering employs genetic engineering techniques to increase production by enhancing gene expression, manipulating a gene's regulatory system (usually transcription factors), preventing branching off to another pathway by down-regulating the competing enzyme, and minimizing catabolism.

The present invention now shows that plants expressing the native bacterial prn operon cloned to the IL-60-based platform produced physiological amounts of the secondary metabolite PRN, sufficient to engender unique phenotypes, without any additional manipulation. PRN production after administration of the prn operon to tomato was at the pmol/mg fresh tissue level, a biologically active level that indeed induced the appearance of unique phenotypes. The obtained IR-PRN construct is transcribed into a polycistronic mRNA, which serves as a template for translation; therefore, it can be defined as an operon. An entire metabolic pathway is expressed, producing the secondary metabolite PRN. Operon-type expression was demonstrated by the appearance of operon-long transcripts, an apparent lack of further processing of those transcripts, the ribosome-bound PRN transcripts being of a size compatible with a full-length polycistronic RNA, and the last gene of the operon indeed being transcribed and translated from an IR-directed transcript. Translation into a polyprotein that would be further processed to maturity and biological activity is inconceivable because of the presence of stop codons and intergenic spaces and because of the fact that not all ORFs are at the same translation frame.

A plant ribosomal binding site (RBS) is present on the IR-PRN construct upstream of prnA. However, the issue of plant ribosome recognition of the other RBSs, in front of each of the downstream ORFs, is currently under study. Mutational analyses clearly demonstrated that expression of all of the operon's genes is required for production of the end product PRN. Taken together, these results indicate that under the control of the IL-60 platform, a plant can express an entire metabolic pathway from an operon in a polycistronic manner. To the best of the inventors' ability to ascertain, the present invention demonstrates for the first time that polycistronic mRNA in a eukaryotic system is a template for translation rather than a pre-mRNA that is further processed to smaller mRNAs.

Furthermore, the present invention now shows that PRN expressing plants are not only protected from pathogen, but also show enhanced growth characteristics leading to increase in crop yield. PRN is known to negatively affect the growth of a wide range of plant pathogens. PRN-producing bacteria have been examined and used for biological control of phytopathogens. The results present herein suggest that when expressed within plant tissues, PRN is protected from the external environment, it is continuously produced, and it does not need to diffuse into the infected tissue to inhibit the invading pathogen. Surprisingly, the PRN compound, found in different parts of the treated plants was not detected in fruit, although the operon itself was detected. However, this phenomenon is of a great advantage as the fruit form the edible commercial crop.

Operon transformation mediated by the IL-60 system presents several advantages over plastid transformation, as described by other authors (Elghabi et al., 2011; Sanz-Barrio et al., 2011; Wei et al., 2011). A comparison between the two operon-expression systems in plants indicates that: 1) the IL-60 system is not transgenic while plastid transformation produces transgenic plants, 2) preparation and handling of the IL-60 system is much simpler than plastid transformation protocols, and 3) IL-60 delivery into plants circumvents the need to use selectable markers. It is conceivable that further manipulation of any of the pathway's signals and genes may elevate the level of PRN production to match that of other manipulated native secondary metabolites.

Methods for introducing the nucleic acids sequences according to the present invention into a plant cell with are known in the art. As used herein the term “introduction” or “introducing” describes a process by which a foreign DNA, such as the expression constructs described herein, enters into a recipient cell and expressed therein. The expression may be stable throughout the plants' life. According to preferred embodiments the nucleic acid sequence of the present invention is transiently transformed into a plant cell, and thus the regenerated plant is not defined as genetically modified organism (non-GMO).

Transient transformation of, for example, leaf cells, meristematic cells, or the whole plant is also envisaged by the present invention. Transient transformation can be effected by any of the direct DNA transfer methods described above or by mechanical or vector mediated viral infection using the plant viruses derived plasmid of the present invention.

According to certain typical embodiments, the expression constructs of the present invention are introduced into the cells of the plant slightly trimmed roots by soaking roots of seedlings or young plantlets in a solution comprising the desired construct or constructs as described in International Application Publication No. WO 2010/004561 to some of the inventors of the present invention.

According to other typical embodiments, the expression constructs of the present invention are introduced into the cells of a plant embryo within a plant seed by soaking the seeds in a seed priming solution comprising the desired construct or constructs as described in International Application Publication No. WO 2011/001434 to some of the inventors of the present invention. According to a particular embodiment, the seeds are intact (e.g. have not been pierced using an injection needle). Further, preferably the seeds which are primed have not undergone imbibition. Preferably the DNA is not taken up by the seeds using agrobacterium tumefaciens. The advantage of using the above-described methods of introduction is in that the expression construct is introduced into an intact plant (when introduced via the roots) or to the embryo from which an intact plant will be developed. Based on the symptomless spreading capability of the expression constructs of the present invention, the introduced prn operon is expressed in vast number of cells of the intact plant without having negative effects on the plant. To the contrary, as disclosed herein, the expression of PRN within the plant results in its enhanced growth.

Alternatively, the expression construct of the present invention can be introduced into isolated cells or tissues by direct DNA transfer. There are various methods of direct DNA transfer into plant cells. In electroporation, the protoplasts are briefly exposed to pulses of strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues. Additional direct DNA transfer techniques include glass or silicon carbide whiskers (see, for example, Dunwell, Methods Mol. Biol. 1999; 111:375-82). The cells or tissues into which the DNA constructs are introduced are them regenerated into plants by methods known to a person skilled in the art.

Following introduction of the PRN operon into the plants, the plants are typically grown under conditions (e.g. light and water) and for a time to allow the plant to flourish (i.e. grow). The plant may be analyzed after 2 days of growth, 3 days of growth, 4 days of growth, 5 days of growth, 6 days of growth, 1 week of growth, 2 weeks of growth, 3 weeks of growth, 1 month of growth or more.

According to one embodiment, the plants are grown in the absence of bacterial or fungal contaminants which are sensitive to PRN. Thus, the plant may be grown in the absence of Rhyzoctonia and/or Botrytis fungi.

Once it is determined that the plants show characteristics of enhanced growth, the plants may be picked and selected.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

Vectors, their Administration to Plants, and Molecular Procedures

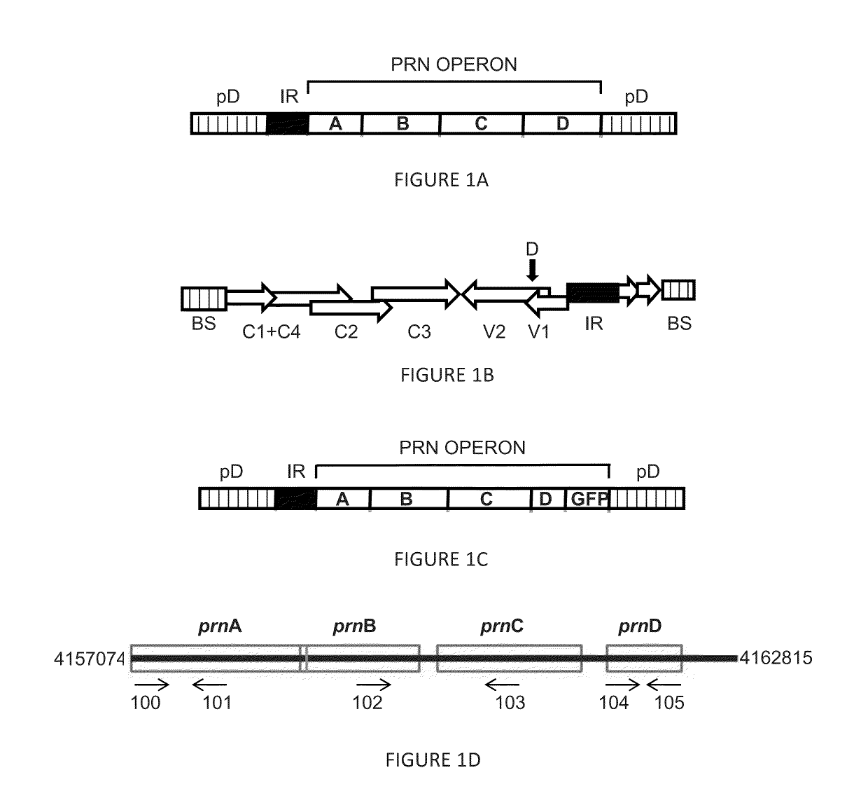

Molecular procedures were carried out according to standard protocols (Sambrook and Russel, 2001. Molecular Cloning, Ed 3. Cold Spring Harbor Laboratory Press, Cold Spring Harbor, N.Y.). The RiboRuler high-range RNA ladder (Fermentas) was used to determine the size of RNA bands in northern-blot analyses. IL-60-BS was described by Peretz et al. (2007, ibid). The IR segment of TYLCV and the following 166 bp of ORF V2 were PCR-amplified with added KpnI and PstI restriction sites and cloned into the plasmid pDrive (Qiagen). The PRN Operon (GenBank accession no. CP000076.1; bases 4,157,074-4,162,815) was inserted into the same plasmid downstream of IR-V2 between the BamHI and XbaI sites. The construct was designated IR-PRN. In another construct, the gene for GFP was fused to prnD of IR-PRN. A clone of tobacco mosaic virus with GFP of improved fluorescence (30B-GFP3; Shivprasad S et al., 1999. Virology 255:312-323) was obtained from Dr. William O. Dawson (University of Florida, Lake Alfred). The coding region of GFP was amplified with primers 106 and 107 (Table 1, SEQ ID NOs:11-12, respectively) carrying restriction sites for BglII and NdeI. Inverse PCR of IR-PRN, starting at both ends of the stop codon of prnD, was performed with primers 104 and 105 carrying restriction sites matching those of the GFP primers (Table 1, SEQ ID NOs; 9-10, respectively). After ligation, the obtained construct carried IR, part of V2, prn (without the prnD stop codon), and the coding region of GFP (initiation ATG was deleted). This enabled the translation of a fused, continuous, prnD-GFP protein. The various constructs and the prn operon are schematically illustrated in FIG. 1. Deletion in prnB was done by cleaving with AjiI and re-ligating. Deletion in prnC was done by cleaving with ScaI and re-ligating. Polyribosomes were prepared as described by Nur et al. (Nur T et al., 1995. J Nutr 125:2457-2462). A cDNA library was prepared from polyribosomal RNA using a Smart cDNA construction kit (Clontech) according to the manufacturer's instructions. The primer for RT was 5′-GCCAGATAGTCATGAATACCTCGCAAAGAG-3′ (SEQ ID NO:6).

TABLE 1
List of primer used

| Primer | Primer sequence | Amplified |
|---|---|---|
| designation | 5′→3′ | Sequence |
| # 101 forward | ATGGATCCATGAACAAGC | Part of prnA |
| | CAATCAAGAATATCGTCA | 838 bp |
| | (SEQ ID NO: 7) | |
| # 101 reverse | GGCCCCAGCATCG | |
| | GAATCTT | |
| | (SEQ ID NO: 8) | |
| # 102 forward | GCGAACGAACACG | Parts of prnB |
| | ATAGCAA | and prnC, |
| | (SEQ ID NO: 9) | 1461 bp |
| # 103 reverse | CGTCAATGAGGGC | |
| | GTGAAT | |
| | (SEQ ID NO: 10) | |
| # 104 forward | ATGAACAACTTCAATTG | The entire |
| | (SEQ ID NO: 11) | prnD |
| # 105 reverse | ATTCTAGACACGAGT | 1091 bp |
| | TGCAACAGCCAGATG | |
| | (SEQ ID NO: 12) | |
| # 106 forward | ATGAGTAAAGGA | The coding |
| | GAAGAACTTTTC | region of GFP |
| | (SEQ ID NO: 13) | (the first ATG |
| # 107 reverse | TTAAAGCTCATC | and 5′UTR |
| | ATGTTTGTATAG | deleted) |
| | (SEQ ID NO: 14) | |
| # 110 Prn | GCCAGATAGTCATGA | Primer for |
| operon | ATACCTCGCAAAGAG | reverse |
| position 6121- | (SEQ ID NO: 15) | transcription |
| 6150 (reverse) | | of ribosomal- |
| | | bound RNA |

Extraction of PRN from Plant Tissue, HPLC, and LC-MS Analyses

Plant tissue (4 g) was taken for each extraction. The protein content in each extract was determined separately. The plant tissue was homogenized in chloroform, and the homogenate was kept at 4° C. for at least 24 h. The mixture was filtered through Miracloth followed by 10 min of centrifugation at 3,000 g. The supernatant fluid was mixed with an equal volume of 0.1 M K₂HPO₄, and after phase separation, the aqueous fraction was discarded. The chloroform fraction was rota-evaporated, and the resultant dry material was dissolved in 100 μl of acetonitrile. Approximately 30 μl of the extract (equivalent to 70 mg of protein in the initial plant extract) was subjected to HPLC separation. HPLC was performed on a 100 RP-18 column (Merck, Lichrospher, 15 μm, 250×4.6 mm). Elution was done in 45% H₂O:30% acetonitrile (GC grade) and 25% methanol or in 65% H₂O:35% acetonitrile. Flow rate was 1 ml/min, and detection was at 225 nm.

In addition, LC-MS analyses were performed on plant extracts. HPLC analysis was performed on an Accela High Speed LC system (Thermo Fisher Scientific Inc.), which consists of an Accela pump, autosampler, and PDA detector. The Accela LC system was coupled with the LTQ Orbitrap Discovery hybrid FT mass spectrometer (Thermo Fisher Scientific Inc.) equipped with an electrospray ionization ion source. The mass spectrometer was operated in negative ionization mode, with the following ion source parameters: spray voltage, 3.5 kV; capillary temperature, 250° C.; and capillary voltage, −35 V; source fragmentation was disabled; sheath gas rate (arb), 30; and auxiliary gas rate, 10. Mass spectra were acquired in the mass-to-charge ratio range of 150 to 2,000 D. The LC-MS system was controlled, and data were analyzed using Xcalibur software (Thermo Fisher Scientific Inc.). The presence of PRN in the sample was confirmed by high resolution LC-MS analysis (measured mass, 254.97284 D; calculated atomic composition of deprotonated pseudomolecular ion C₁₀H₅O₂N₂³⁵Cl₂, error −2.0 μg/ml).

Bioassay for Inhibition of Rhizoctonia solani In Vitro

R. solani was cultured on potato dextrose agar at 28° C. A disk (30 mm in diameter) of R. solani-containing agar was transferred to another Petri dish containing potato dextrose agar and 10 μl of HPLC elution buffer (negative control), 10 μl of various plant extracts, or 10 μl containing 0.2 mg of synthetic PRN in elution buffer (Sigma, positive control). Inhibition of fungal growth was determined after incubation at 28° C. for 3 days.

Assay for R. solani Resistance

Roots of tomato (Solanum lycopersicum) seedlings were immersed in a water solution of IL-60-BS and IR-PRN or IR-PRN-GFP (1 μg/plantlet) until the entire solution was sucked up by the plants. The plants were then potted and kept in the greenhouse at 24° C. Tomato seedlings, 2 weeks after application of IL-60-BS and IR-PRN, were transferred to pots with 0.5 kg of soil mixed with 0.5 g of R. solani mycelium. Control plants (not carrying PRN) were similarly treated. Plants were kept for 2 to 3 weeks at 24° C. until symptom appearance.

### Growth Assay

Seeds of tomato (Solanum lycopersicum) cv Moneymaker were primed with a priming solution containing 25% PEG without (control) or with IR-PRN.

The seeds were sown in randomly placed small tagged pots containing potting mixture, in a glasshouse at a night and day temperature of 15-18° C. and 22-27° C., respectively. Plant height, weight and dry weight were measured when the plant reached 3-5 true leaves. Typically the seedlings were 4-5 weeks old.

### Confocal Microscopy

Tomato leaf sections, after peeling away the epidermis, were observed under the confocal microscope (Zeiss 100M). Excitation was at 488 nm. GFP emission was detected at 505 to 550 nm. Autofluorescence of chlorophyll was detected at wavelengths greater than 560 nm. Data were processed by the built-in program LSM 51.

### Example 1

### Delivery, Replication, Expression, and Spread of the prn Operon in Tomato Plants

The components of the plant universal vector IL-60 employed throughout this study are described in Peretz et al. (2007, ibid). Briefly, a plasmid was inserted into the replication-association gene of TYLCV, disabling rolling-circle replication but maintaining replication from double stranded DNA to double-stranded DNA, which is directed solely by host factors. Any DNA placed downstream of the viral IR that carries the origin of replication and two bidirectional promoters will replicate in the cells into which it has been delivered. However, to spread to other cells, it requires a helper virus or IL-60-BS, which promotes movement throughout the plant without causing disease. All constructs were administered to the plants by root uptake. The root tips of young seedlings were slightly trimmed and immersed in an aqueous solution containing 1 mg of each plasmid per plant. Tap water was added when the solution was fully absorbed by the plants and the plants were immersed for 3 days, after which they were planted in soil.

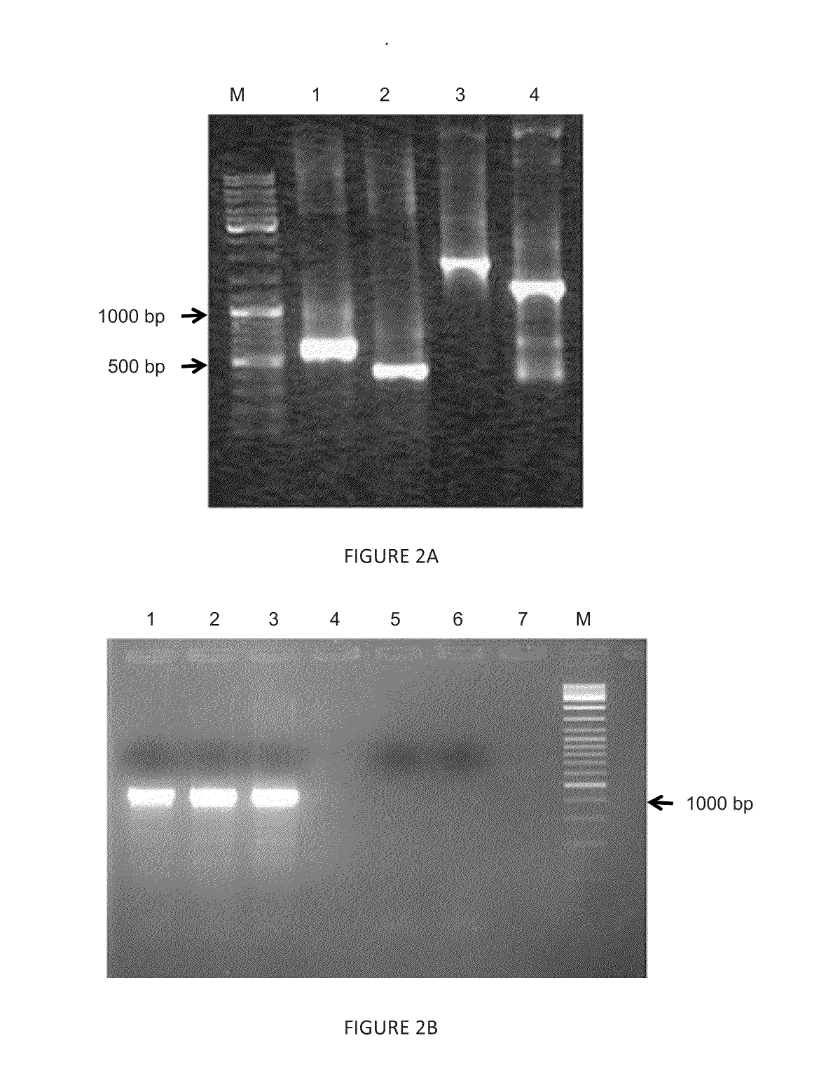

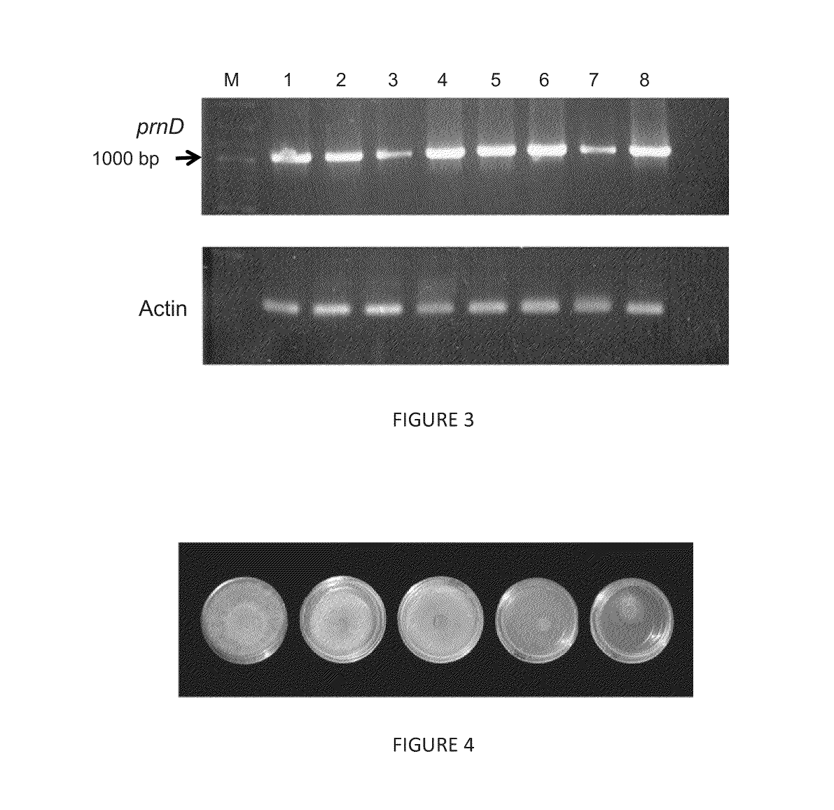

The entire prn operon of P. fluorescens strain Pf-5 (GenBank accession no. CP000076.1; bases 4,157,074-4,162,815) was cloned in the plasmid pD-IR and was designated IR-PRN. IR-PRN was administered to plants along with the IL-60-BS “driver” as described in the Materials and Methods section hereinabove. IR-PRN replication and spread in the plant was determined by PCR of DNA extracted from leaves and other plant organs. Two weeks after administration to plants, prn DNA was found in all tested tissues, including flowers and fruit at flowering and fruit set (FIG. 2). Transcription from IR-PRN was determined by reverse transcription (RT)-PCR (FIG. 3) by analyzing ribosome-bound RNA (as cDNA) and by northern blot analysis (described below).

### Example 2

### Production of Biologically Active PRN by Plants Harboring the prn Operon

In bacteria, the end product of the prn operon is PRN. Extracts of various plant organs were analyzed by HPLC for the presence of PRN, as described in the “Materials and Methods” section hereinabove. HPLC samples were adjusted for equal protein content. PRN was found in extracts of roots and leaves of plants carrying IR-PRN but not in fruits. Liquid chromatography (LC)-mass spectrometry (MS) analysis identified a peak in IR-PRN treated plants that was absent in control plants, corresponding in mass (254.97 D) to PRN (data not shown).

PRN is an antifungal, antibacterial compound that inhibits the growth of a wide spectrum of plant pathogens. One of the affected pathogens is the fungus Rhizoctonia solani. Therefore, the various HPLC-eluted fractions were tested for their capacity to inhibit the growth of R. solani. Biological tests showed that the plant-extracted, HPLC-purified PRN was indeed inhibitory to R. solani (FIG. 4). It was concluded that the plants produced PRN identical to that produced in bacteria by the same operon.

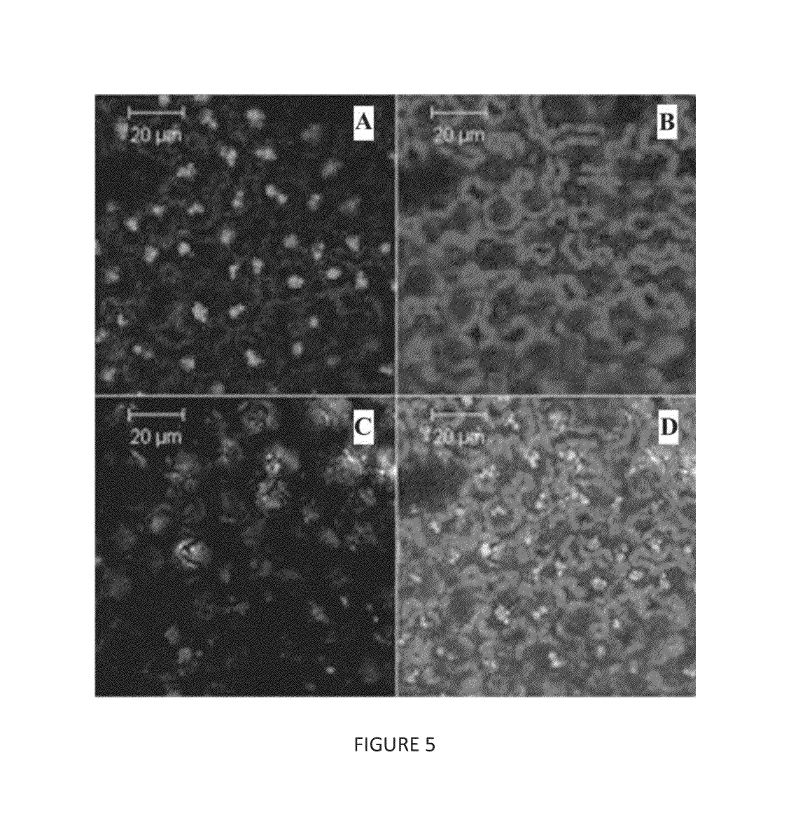

Furthermore, prn expression in plants is shown to be polycistronic. To determine whether, in addition to prnA (which carries a viral RBS in front of it), the other three ORFs are also required for PRN production and are not replaced by functionally equivalent plant proteins, we mutated those genes. We first tested whether translation of the most 3′-distal ORF (prnD) originates from the IR-directed construct and not from a gene directed by a plant promoter. We fused GFP (the 5′-untranslated region was deleted to remove predicted RBSs, and the first ATG was omitted to prevent possible translation initiation at the start of the GFP coding region) to prnD to produce IR-PRN-GFP and then administered this construct to plants. GFP fluorescence in these plants indicated that prnD was translated from IR-directed polycistronic mRNA and was not of plant origin. The protein PRND-GFP appeared to be secreted into the vacuole and to aggregate (FIG. 5).

A series of controlled experiments indicated that all four genes of the PRN operon are required for PRN production in plants. Plants carrying IR-PRN with deletions in prnB or prnC did not produce PRN. Plants expressing IR-PRN-GFP did not produce PRN (probably due to inactivation of the GFP-fused protein PRND). Plants in which PRN had been replaced by an irrelevant gene (GUS) or TYLCV-infected plants did not produce PRN (data not shown). Only the positive controls exhibited PRN (elution time of 27.9 min). PRN could not be detected by HPLC in fruit, even though the fruit harbored IR-PRN-DNA (FIG. 2B), probably due to interference in expression.

### Example 3

### PRN-Expressing Plants are Resistant to Damping-Off Disease Caused by R. solani

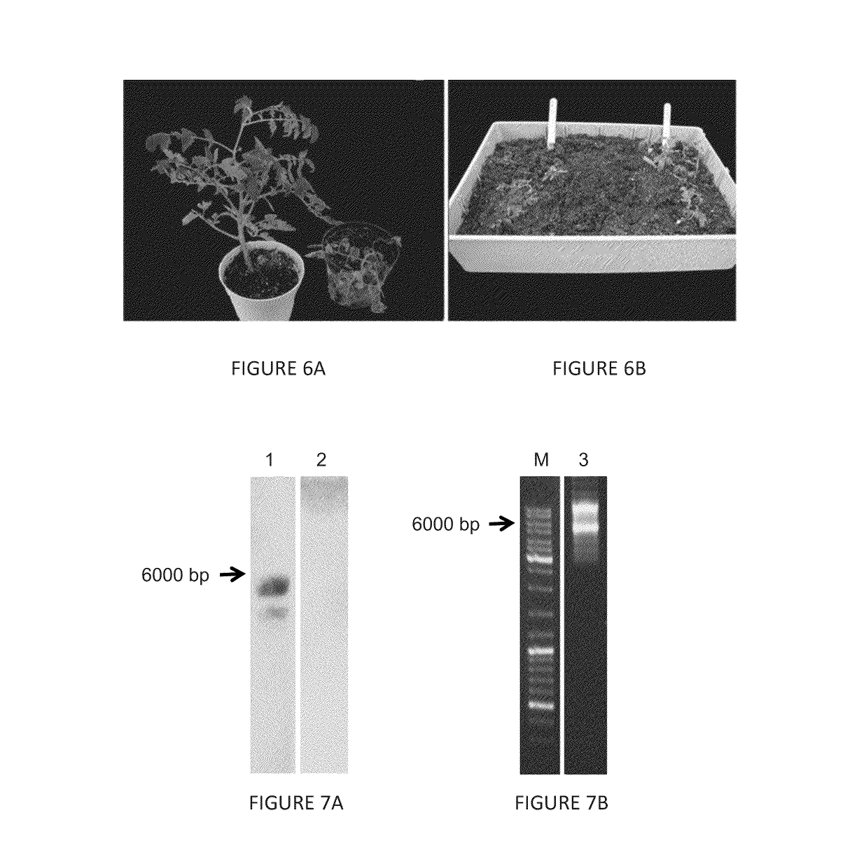

Because PRN is inhibitory to R. solani and was produced in all plant tissues except fruits, the PRN expressing plants were tested for resistance to damping off disease of tomato caused by R. solani. Young control and PRN-expressing tomato plants were planted in pots containing soil mixed with R. solani mycelium. The control plants, which did not express PRN, wilted within 2 weeks, whereas the PRN-expressing plants remained healthy (FIG. 6). Various PRN-producing bacteria have been tested by others for biological control of phytopathogens, but despite their potential antifungal activity, results have been inconsistent (e.g. Compant S. et al., 2005. Appl Environ Microbiol 71:4951-4959). In contrast, as shown herein, 80% to 100% of the PRN expressing plants were protected from infection.

### Example 4

### PRN-Expressing Plants Produce Operon-Long Transcripts

A true polycistronic mode of expression has not, to the best of the inventors' knowledge, been demonstrated for eukaryotes. To determine whether the bacterial operon is correctly expressed in plants, northern-blot analyses were performed. FIG. 7 (lanes 1 and 2) shows that the prn operon was transcribed into two long transcripts (the size of the major transcript is 5.5-6 kb, and the shorter one is approximately 4-5 kb, probably due to different termination signals). In addition, polyribosomes from PRN expressing plants were isolated, cDNA from the ribosomal bound RNA was prepared and long cDNA (5-6 kb and p) was amplified by PCR with a prn-specific primer (FIG. 7, lane 3). Two major cDNA species were observed: One was 5 to 6 kb long, and the other was longer (possibly due to incomplete 3′ trimming of the nascent transcript). Nevertheless, smaller-size prn transcripts were not detected, indicating a lack of further processing to mono-cistronic mRNAs. Hence, the poly-cistronic long transcript itself, rather than processed mature RNAs, served as the template for translation, as is the case in prokaryotes.

### Example 5

### PRN-Expressing Plants Show Enhanced Growth Pattern

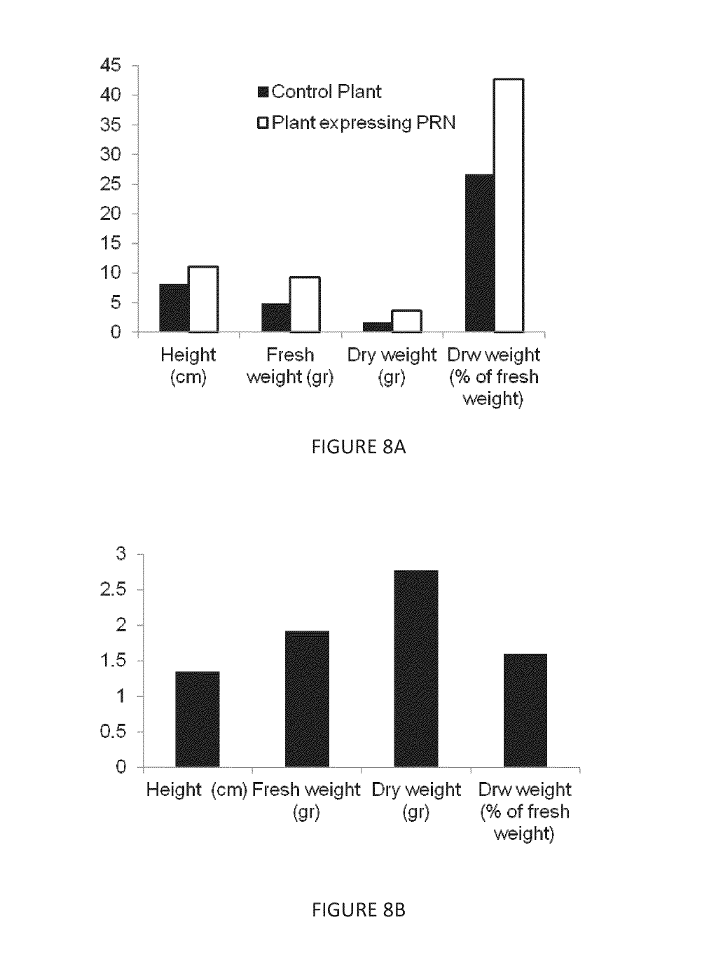

The inventors of the present invention have previously found that introducing an exogenous DNA construct into plant embryo via imbibing seeds in modified priming medium has no deleterious effect of the embryo growth (data not shown). As described hereinabove, introducing the entire prn operon into tomato cells resulted in significant expression of PRN. Evidently, the tomato seedling expressing PRN showed enhanced growth as compared to the growth of control plants not harboring the prn operon. The enhancement was observed in all parameters examined, including plant height, wet and dry weight (Table 2 and FIG. 8).

TABLE 2
Growth enhancement by PRN

| | | Plant expressing | Growth |
|---|---|---|---|
| Growth Measure | Control Plant | PRN | Enhancement (%) |
| Height (cm) | 8.2 | 11 | 134.1 |
| Fresh weight(gr) | 4.8 | 9.2 | 191.7 |
| Dry weight (gr) | 1.6 | 3.6 | 276.9 |
| Dry weight (% of | 26.7 | 42.7 | 159.9 |
| fresh weight) | | | |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A method for enhancing the growth of a crop plant, comprising introducing into at least one cell of a plant at least one virus-based expression construct comprising heterologous DNA comprising the entire prn operon thereby enhancing the growth of the plant compared to the growth of a corresponding control plant.

2. The method of claim 1, further comprising analyzing the amount of crop yield or biomass of the plant.

3. The method of claim 2, wherein said analyzing is effected by measuring an expression of a growth-related gene.

4. The method of claim 2, wherein said analyzing is effected by measuring a weight or a volume of said crop yield of the plant.

5. The method of claim 4, wherein said analyzing is effected by measuring a weight or a number of seeds of the plant.

6. The method of claim 1, wherein the growth of the plant is effected in the absence of a bacterial or fungal contaminant sensitive to said PRN.

7. The method of claim 1, wherein the virus-based expression construct comprises viral genes or parts thereof enabling replication and symptomless spreading of the construct into adjacent plant cells.

8. The method of claim 7, wherein the virus-based expression construct is a Geminivirus based construct.

9. The method of claim 8, wherein the virus-based expression construct comprises the entire prn operon downstream to a non-contiguous nucleic acid sequences of Geminivirus intergenic region (IR).

10. The method of claim 8, wherein the Geminivirus is Tomato Yellow Leaf Curl Virus (TYLCV).

11. The method of claim 1, said method comprises introducing into the plant cell a single TYLCV based expression construct comprising the entire prn operon, wherein the construct enables replication and symptomless spreading of said construct into adjacent plant cells.

12. The method of claim 1, said method comprises co-introducing into the plant cell a first TYLCV based expression construct comprising the entire prn operon and viral genes or parts thereof enabling replication confined to the plant cell and a second TYLCV based expression construct comprising viral genes or parts thereof enabling symptomless spreading of at least the first construct from said plant cell into adjacent plant cells.

13. The method of claim 12, wherein the first DNA construct comprises the entire prn operon downstream of non-contiguous TYLCV viral intergenic region (IR).

14. The method of claim 13, wherein the TYLCV viral intergenic region (IR) is followed by the 5′ terminal sequence of TYLCV V2.

15. The method of claim 14, wherein TYLCV viral intergenic region (IR) comprises the nucleic acid sequence set forth in SEQ ID NO:3 and the 5′ terminal sequence of TYLCV V2 comprises the nucleic acid sequence set forth in SEQ ID NO:4.

16. The method of claim 8, wherein the least one Geminivirus-based expression construct is introduced into at least one cell of a plant root by placing roots of the plant in a solution comprising said at least one Geminivirus-based expression construct so as to allow said construct to be introduced into the root cell.

17. The method of claim 8, wherein the at least one Geminivirus-based expression construct is introduced into at least one cell of a plant embryo by priming a plant seed which has not undergone imbibition using a priming medium containing said at least one Geminivirus-based expression construct under conditions which allow uptake of the DNA by said plant seed.

18. The method of claim 1, wherein enhancing the growth comprises at least one of increasing the plant height, increasing the plant fresh weight, increasing the plant dry weight and increasing the plant crop yield.

19. A plant produced by the method of claim 1.

20. A plant comprising at least one cell comprising at least one Geminivirus based expression construct, the construct comprising an entire prn operon downstream to a non-contiguous nucleic acid sequences of Geminivirus intergenic region (IR), wherein the plant shows enhanced growth compared to a corresponding control plant.

21. The plant of claim 20, wherein the Geminivirus-based construct is selected from the group consisting of IL-60 having the nucleic acids sequence set forth on SEQ ID NO:1 further comprising the prn operon; IL-60-BS having the nucleic acids sequence set forth in SEQ ID NO:2 further comprising the prn operon; IR-PRN comprising the nucleic acid sequence set forth in SEQ ID NOs:3 and 4 further comprising the prn operon; and combinations thereof.

22. The plant of claim 20, wherein the prn operon is the entire prn operon of Pseudomonas fluorescens strain Pf-5 (GenBank accession No. CP000076.1; bases 4,157,074-4,162,815).

23. The plant of claim 20, said plant shows at least one of increased height, increased fresh weight and increased dry weight compared to a corresponding control plant.

24. The plant of claim 23, said plant has an increased crop yield compared to a corresponding control plant.
